(19) United States
(12) Patent Application Publication
(10) Pub. No.: US 2025/0163027 A1
(43) Pub. Date: May 22, 2025

(54) GPR88-RECEPTOR-AGONIST

(71) Applicant: Boehringer Ingelheim International GmbH, Ingelheim am Rhein (DE)

(72) Inventors: Julian KLEPP, Biberach (DE); Vincent FAYE, Illkirch-Graffenstaden (FR); Mickaël FER, Ostwald (FR); Christoph HOENKE, Biberach an der Riss (DE); Oliver HUCKE, Warthausen (DE); Louison LEBRUN, Illkirch-Graffenstaden (FR); Stanislas MAYER, Eschau (FR); Florian MONTEL, Mietingen (DE); Laurent SCHAEFFER, Fortschwihr (FR)

(21) Appl. No.: 18/951,962

(22) Filed: Nov. 19, 2024

Related U.S. Application Data
(60) Provisional application No. 63/600,842, filed on Nov. 20, 2023.

(51) Int. Cl. C07D 403/12; A61K 31/506

(52) U.S. Cl. CPC C07D 403/12; A61K 31/506

(57) ABSTRACT

The present invention relates to a novel N-[(Pyrimidinylamino) propanyl]-arylcarboxamide derivative, its pharmaceutical compositions containing it and its use in therapy, particularly in the treatment or prevention of conditions having an association with the GPR88-receptor.

### FIELD OF THE INVENTION

The present invention relates to (1R,2R)—N-(2-(difluoromethoxy)ethyl)-N-(4-(4-isopropoxyphenyl)-1-methyl-1H-indazol-7-yl)-2-(pyrimidin-4-yl) cyclopropane-1-carboxamide, its pharmaceutical compositions containing it and its use in therapy, particularly in the treatment or prevention of conditions having an association with the GPR88 receptor.

### BACKGROUND OF THE INVENTION

GPR88 is a G-protein-coupled receptor (GPR) expressed in the central nervous system (CNS) [K. Mizushima, Y. Miyamoto, F. Tsukahara, M. Hirai, Y. Sakaki, T. Ito, Genomics 2000, 69, 314-321; V. V. Waes, K. Y. Tseng, H. Steiner, Basal Ganglia 2011, 1, 83-89; R. Massart, J. Guilloux, V. Mignon, P. Sokoloff, J. Diaz, Eur J Neurosci 2009, 30, 397-414; H. Komatsu, Int. J. Mol. Sci. 2015, 16, 14109-14121]. It was originally identified in the striatum but is also expressed in the cerebral cortex, amygdala, and hypothalamus among other brain regions [M. Ubaldi, E. Ricciardelli, L. Pasqualini, G. Sannino, L. Soverchia, B. Ruggeri, S. Falcinelli, A. Renzi, C. Ludka, R. Ciccocioppo, G. Hardiman, Pharmacogenomics 2015, 16, 471-482; Ghate, K. Befort, J. A. J. Becker, D. Filliol, C. Bole-Feysot, D. Demebele, B. Jost, M. Koch, B. L. Kieffer, Neuroscience 2007, 146, 1182-1192; A. C. Meirsman, J. L. Merrer, L. P. Pellissier, J. Diaz, D. Clesse, B. L. Kieffer, J. A. J. Becker, Biol Psychiat 2016, 79, 917-927; R. Massart, V. Mignon, J. Stanic, P. Munoz-Tello, J. A. J. Becker, B. L. Kieffer, M. Darmon, P. Sokoloff, J. Diaz, J Comp Neurol 2016, 524, 2776-2802; T. M. Arefin, A. E. Mechling, A. C. Meirsman, T. Bienert, N. S. Hübner, H.-L. Lee, S. B. Hamida, A. Ehrlich, D. Roquet, J. Hennig, D. von Elverfeldt, B. L. Kieffer, L.-A. Harsan, Brain Connectivity 2017, 7, 526-540]. GPR88 plays a role in learning, emotional responses, and social behavior in many species. [J. Lau, A. Farzi, R. F. Enriquez, Y.-C. Shi, H. Herzog, Sci Rep-uk 2017, 7, 9912; D. M. Thomson, R. L. Openshaw, E. J. Mitchell, M. Kouskou, M. J. Millan, C. M. la Cour, B. J. Morris, J. A. Pratt, Genes Brain Behav 2021, 20, e12710; M. Ingallinesi, L. L. Bouil, N. F. Biguet, A. D. Thi, C. M. la Cour, M. J. Millan, P. Ravassard, J. Mallet, R. Meloni, Mol Psychiatr 2015, 20, 951-958; A. T. Ehrlich, M. Semache, J. Bailly, S. Wojcik, T. M. Arefin, C. Colley, C. L. Gouill, F. Gross, V. Lukasheva, M. Hogue, E. Darcq, L.-A. Harsan, M. Bouvier, B. L. Kieffer, Brain Struct Funct 2018, 223, 1275-1296]. Aberrant GPR88 activity is associated with many behavior-linked neurological disorders such as bipolar disorder, schizophrenia, speech delay, learning disabilities major psychoses and attention deficit hyperactivity disorder (ADHD), as well as response to psychostimulant drugs, alcohol, and antidepressants. [A. C. Meirsman, A. Robé, A. de K. d′Exaerde, B. L. Kieffer, Eneuro 2016, 3, ENEURO. 0202-16.2016; A. T. Ehrlich, G. Maroteaux, A. Robe, L. Venteo, Md. T. Nasseef, L. C. van Kempen, N. Mechawar, G. Turecki, E. Darcq, B. L. Kieffer, Commun Biology 2018, 1, 102; M. D. Zompo, J. Deleuze, C. Chillotti, E. Cousin, D. Niehaus, R. P. Ebstein, R. Ardau, S. Mace, L. Warnich, M. Mujahed, G. Severino, C. Dib, E. Jordaan, I. Murad, S. Soubigou, L. Koen, I. Bannoura, C. Rocher, C. Laurent, M. Derock, N. F. Biguet, J. Mallet, R. Meloni, Mol Genetics Genom Medicine 2014, 2, 152-159; S. B. Hamida, M. Carter, E. Darcq, M. Sourty, M. T. Rahman, A. M. Decker, C. Jin, B. L. Kieffer, Addict. Biol. 2022, 27, e13227; F. Alkufri, A. Shaag, B. Abu-Libdeh, O. Elpeleg, Neurol.: Genet. 2016, 2, NA; S. F. Logue, S. M. Grauer, J. Paulsen, R. Graf, N. Taylor, M. A. Sung, L. Zhang, Z. Hughes, V. L. Pulito, F. Liu, S. Rosenzweig-Lipson, N. J. Brandon, K. L. Marquis, B. Bates, M. Pausch, Mol Cell Neurosci 2009, 42, 438-447].

Several studies have highlighted the receptor's central role in neurological function and potential as a therapeutic target for neurological and psychiatric disorders [C. Jin, A. M. Decker, T. L. Langston, Bioorgan Med Chem 2017, 25, 805-812; C. Jin, A. M. Decker, V. H. Makhijani, J. Besheer, E. Darcq, B. L. Kieffer, R. Maitra, J Med Chem 2018, 61, 6748-6758; C. Jin, A. M. Decker, D. L. Harris, B. E. Blough, Acs Chem Neurosci 2016, 7, 1418-1432. S. B. Hamida, S. Mendonça-Netto, T. M. Arefin, Md. T. Nasseef, L.-J. Boulos, M. McNicholas, A. T. Ehrlich, E. Clarke, L. Moquin, A. Gratton, E. Darcq, L. A. Harsan, R. Maldonado, B. L. Kieffer, Biol Psychiat 2018, 84, 202-212; Y. Bi, C. D. Dzierba, C. Fink, Y. Garcia, M. Green, J. Han, S. Kwon, G. Kumi, Z. Liang, Y. Liu, Y. Qiao, Y. Zhang, G. Zipp, N. Burford, M. Ferrante, R. Bertekap, M. Lewis, A. Cacace, R. S. Westphal, D. Kimball, J. J. Bronson, J. E. Macor, Bioorg Med Chem Lett 2015, 25, 1443-1447; C. Jin, A. M. Decker, X.-P. Huang, B. P. Gilmour, B. E. Blough, B. L. Roth, Y. Hu, J. B. Gill, X. P. Zhang, Acs Chem Neurosci 2014, 5, 576-587]. Notably, one study in GPR88 knockout mice (KO) demonstrated a schizophrenic-like phenotype and dopamine D2 receptor hypersensitivity in GPR88 KO, which manifested as increased sensitivity to apomorphine-induced climbing and stereotypy and amphetamine-stimulated locomotor activity [F. Alkufri, A. Shaag, B. Abu-Libdeh, O. Elpeleg, Neurol.: Genet. 2016, 2, NA;]. In another, the animals exhibited increased locomotion, diminished motor coordination, and impaired cue-based learning [A. Quintana, E. Sanz, W. Wang, G. P. Storey, A. D. Güler, M. J. Wanat, B. A. Roller, A. L. Torre, P. S. Amieux, G. S. McKnight, N. S. Bamford, R. D. Palmiter, Nat Neurosci 2012, 15, 1547-1555]. GPR88 re-expression normalized these behaviors, which may signify that GPR88 dysfunction contributes to abnormal behaviors observed for neurological and psychiatric diseases.

Upregulation of GPR88 function for the treatment of neurological disease requires the use of either agonists or positive allosteric modulators (PAM). Of note, a 2019 patent from Pandeia disclosed compounds with reasonable potency without the use of a primary amine, which is associated with poor p-glycoprotein (PGP) efflux [WO2022/129933]. In recent years, Jin et al., published a series a medicinal chemistry articles optimizing the Structure—Activity Relationship (SAR) of a series of GPR88 agonists [C. Jin, A. M. Decker, T. L. Langston, Bioorgan Med Chem 2017, 25, 805-812; C. Jin, A. M. Decker, V. H. Makhijani, J. Besheer, E. Darcq, B. L. Kieffer, R. Maitra, J Med Chem 2018, 61, 6748-6758; C. Jin, A. M. Decker, D. L. Harris, B. E. Blough, Acs Chem Neurosci 2016, 7, 1418-1432; M. T. Rahman, A. M. Decker, L. Laudermilk, R. Maitra, W. Ma, S. B. Hamida, E. Darcq, B. L. Kieffer, C. Jin, J Med Chem 2021, DOI 10.1021/acs.jmedchem.1c01075; M. T. Rahman, A. M. Decker, S. B. Hamida, D. A. Perrey, H. H. C. Lakmal, R. Maitra, E. Darcq, B. L. Kieffer, C. Jin, J. Medicinal Chem. 2023, 66, 2964-2978]. In their explorations, Jin et al. demonstrated that both compounds could attenuate binge-like alcohol drinking in a murine model. Altogether, the literature suggests a crucial role for GPR88 in the regulation of striatal function.

### DETAILED DESCRIPTION OF THE INVENTION

The present invention provides a novel N-[(Pyrimidinylamino) propanyl]-arylcarboxamide derivative, namely the compound (1R,2R)—N-(2-(difluoromethoxy)ethyl)-N-(4-(4-isopropoxyphenyl)-1-methyl-1H-indazol-7-yl)-2-(pyrimidin-4-yl) cyclopropane-1-carboxamide:

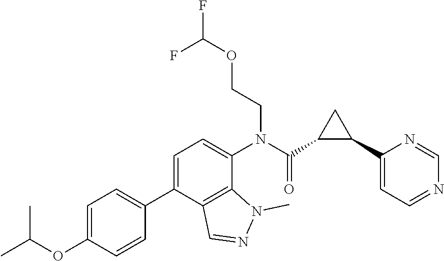 [SMILES: CC(C)Oc1ccc(-c2ccc(N(CCOC(F)F)C(=O)[C@@H]3C[C@H]3c3ccncn3)c3c2cnn3C)cc1]

which is a potent GPR88 agonist (assay A).

In addition, the present invention provides a pharmaceutical composition comprising the compound of the present invention, or a pharmaceutically acceptable thereof, and a pharmaceutically acceptable adjuvant, diluent and/or carrier.

The present is further direct to a method for treating a psychiatric or neurological condition associated with impulse control deficits, addiction, speech delay, learning disabilities, depression, psychosis, bipolar disorder and/or emotional valence in a patient comprising administering to said patient a therapeutically effective amount of a compound of the present invention, or a pharmaceutically acceptable salt thereof. In another embodiment, the psychiatric or neurological condition to be treated is selected from the group consisting of major depressive disorder, Huntington's disease, addiction, Parkinsons's disease, attention deficit hyperactivity disorder, borderline personality disorder, post-traumatic stress disorder, attention deficit hyperactivity disorder and cognitive impairments associated with schizophrenia.

### General Definitions

Terms not specifically defined herein should be given the meanings that would be given to them by one skilled in the art considering the disclosure and the context.

### Stereochemistry:

Unless specifically indicated, throughout the specification and the appended claims, a given chemical formula or name shall encompass tautomers and all stereo, optical and geometrical isomers (e.g. enantiomers, diastereomers, E/Z isomers etc.) and racemates thereof, as well as mixtures in different proportions of the separate enantiomers, mixtures of diastereomers, or mixtures of any of the foregoing forms where such isomers and enantiomers exist, as well as salts, including pharmaceutically acceptable salts thereof.

### Salts:

The phrase “pharmaceutically acceptable” is employed herein to refer to those compounds, materials, compositions, and/or dosage forms which are, within the scope of sound medical judgment, suitable for use in contact with the tissues of human beings without excessive toxicity, irritation, allergic response, or other problem or complication, and commensurate with a reasonable benefit/risk ratio.

As used herein, “pharmaceutically acceptable salts” refer to derivatives of the disclosed compounds wherein the parent compound forms a salt with an acid. Examples for acids forming a pharmaceutically acceptable salt with a parent compound containing a basic moiety include mineral or organic acids such as benzenesulfonic acid, benzoic acid, citric acid, ethanesulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid or tartaric acid.

The pharmaceutically acceptable salts of the present invention can be synthesized from the parent compound which contains a basic or acidic moiety by conventional chemical methods. Generally, such salts can be prepared by reacting the free acid or base forms of these compounds with a sufficient amount of the appropriate base or acid in water or in an organic diluent like ether, ethyl acetate, ethanol, isopropanol, or acetonitrile, or a mixture thereof. Salts of other acids than those mentioned above which for example are useful for purifying or isolating the compounds of the present invention (e.g. trifluoroacetate salts) also comprise a part of the invention.

### BIOLOGICAL ASSAYS

### Abbreviations:

aq. aqueous
ACN acetonitrile
Ar argon
BSA bovine serum albumin
CyHex cyclohexane
DCM dicholoromethane
DMEM Dulbecco's Modified Eagle's Medium
DMF N,N-dimethylformamide
DMSO dimethyl sulfoxide
EA ethyl acetate
ESI-MS electrospray ionisation mass spectrometry
ebBRET enhanced bystander bioluminescence resonance energy transfer
EMTA effector membrane translocation assay
eq. equivalent(s)
h hour(s)
HEPES 4-(2-hydroxyethyl)-1-piperazineethanesulfonic acid
HPLC high-performance liquid chromatography
HPLC-MS high-performance liquid chromatography-mass spectrometry
HRMS high-resolution mass spectrometry
M molarity
MeOH methanol
min minutes
MS mass spectrometry
N Normality
NBS N-bromosuccinimide
NMP N-methyl-2-pyrrolidone
PE petroleum ether
PEI polyethylenimine
Rlucll Renilla luciferase
RT room temperature
Rt retention time
THB Tyrode-Hepes buffer

The biological activity of compounds is determined by the following methods:

A. In Vitro Testing of GPR88: hGPR88 Gi1 BRET Assay

GPR88 was previously shown to be coupled to the Gi/o protein family [A. Quintana, E. Sanz, W. Wang, G. P. Storey, A. D. Güler, M. J. Wanat, B. A. Roller, A. L. Torre, P. S. Amieux, G. S. McKnight, N. S. Bamford, R. D. Palmiter, Nat Neurosci 2012, 15, 1547-1555; C. Jin, A. M. Decker, X.-P. Huang, B. P. Gilmour, B. E. Blough, B. L. Roth, Y. Hu, J. B. Gill, X. P. Zhang, Acs Chem Neurosci 2014, 5, 576-587]. To detect the activation of Gi1 protein subtype, we used an effector membrane translocation assay (EMTA) biosensor platform [C. Avet, A. Mancini, B. Breton, C. L. Gouill, A. S. Hauser, C. Normand, H. Kobayashi, F. Gross, M. Hogue, V. Lukasheva, S. St-Onge, M. Carrier, M. Héroux, S. Morissette, E. B. Fauman, J.-P. Fortin, S. Schann, X. Leroy, D. E. Gloriam, M. Bouvier, eLife 2022, 11, e74101] based on enhanced bystander bioluminescence resonance energy transfer (ebBRET) [Y. Namkung, C. L. Gouill, V. Lukashova, H. Kobayashi, M. Hogue, E. Khoury, M. Song, M. Bouvier, S. A. Laporte, Nat. Commun. 2016, 7, 12178]. This biosensor consists of the sub-domain of the G protein-effector protein Rap1GAP that selectively interacts with activated Gi/o. This domain was fused at C-terminus to Renilla luciferase (Rlucll) and co-expressed with alpha subunit of Gi1 protein and unmodified GPR88 receptor. Upon GPCR activation, the energy donor-fused effector translocates to the plasma membrane to bind activated Ga protein, bringing Rlucll in close proximity to the energy acceptor, Renilla green fluorescent protein, targeted to the plasma membrane through a CAAX motif (rGFP-CAAX), thus leading to an increase in ebBRET. All newly synthesized compounds were tested on human GPR88. The plasmid encoding human GPR88 receptor (natural variant Val190lle) was produced by codon-optimized gene synthesis based on reference sequence (NM_022049.2:c.568G>A). Using the same EMTA Gi1 biosensor platform, selected compounds were also tested on mouse and rat GPR88 (using codon-optimized gene synthesis based on reference sequences NM_022427.2 and NM_031696.2, respectively), as well as on counter-screen on another Gi1-coupled receptor, namely human alpha2A adrenergic receptor (using codon-optimized gene synthesis based on reference sequence NM_000681.3) in order to evaluate their specificity. The ebBRET experiments were performed on HEK293 cells transiently transfected with plasmid DNAs encoding biosensor, receptor, and G protein using polyethylenimine (PEI) as a transfection agent, as previously described (Avet C, eLife, 2022). The combination of DNA/PEI mixture and cell suspension was seeded into F75 cell culture flasks, which were incubated at 37° C., 5% CO2 for 48 h in culture medium (DMEM+10% FBS+penicillin/streptomycin 1%). At 48 h post-transfection, cell layers were rinsed once with PBS and detached using Trypsin-EDTA solution. Cells were harvested, counted, centrifuged, and resuspended in Tyrode-Hepes buffer (THB). White 384-well plates were inoculated with 20 μL/well (20,000 cells/well). Cells were then equilibrated in the dark for 1 hr at room temperature (+22° C.). The GPR88 agonist compounds were tested over a range of 20 concentrations, ranging from 100 μM to 0.003 μM. To proceed with cell stimulation, compounds were dispensed in columns 3 to 22 of 384 well plates previously seeded with transfected cells, using digital dispenser HP D300e from TECAN; positive (2-PCCA) and negative (DMSO vehicle) controls were dispensed into the wells of columns 1, 2 and 23, 24, respectively. The assay plates were incubated for 10 minutes at room temperature. BRET signal was induced by addition of the luciferase substrate Prolume Purple in each well to a final concentration of 3 μM. Dual emissions at 410 nm (BW 80 nm) and 515 nm (BW 30 nm) were measured on multi-mode microplate reader EnVision (Perkin Elmer) with an integration time of 500 msec. The analysis of the data was performed by the calculation of the BRET ratio of light emitted by GFP-acceptor (515 nm) over light emitted by luciferase-donor (410 nm).

### Biological Data

### The Compound of the Present Invention

A comparison of the GPR88 potencies of the compound of the present invention with exemplary prior art compounds is shown in Table 1.

TABLE 1
Biological data of the compound of the present invention vis a vis the exemplary prior
art compounds

| | | Assay A |
|---|---|---|
| | | BRET |
| | | hGPR88 |
| | | receptor |
| Example | Structure | [nM] |
| 1 | 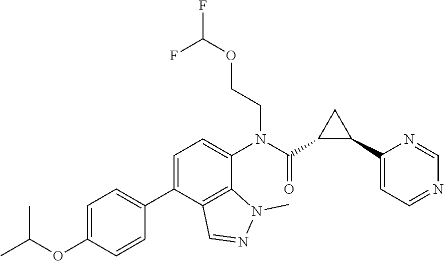 [SMILES: CC(C)Oc1ccc(-c2ccc(N(CCOC(F)F)C(=O)[C@@H]3C[C@H]3c3ccncn3)c3c2cnn3C)cc1] | 47 |
| RTI- 13951-33 in J Med Chem 2018, 61, 6748- 6758 | 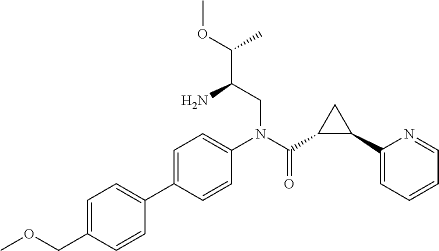 [SMILES: COCc1ccc(-c2ccc(N(C[C@@H](N)[C@@H](C)OC)C(=O)[C@@H]3C[C@H]3c3ccccn3)cc2)cc1] | 228 |
| Ex 141 in WO2022/ 129933 | 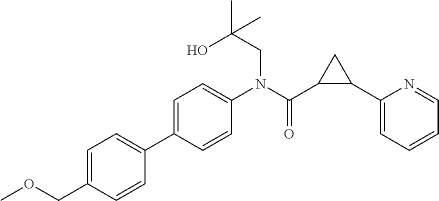 [SMILES: COCc1ccc(-c2ccc(N(CC(C)(C)O)C(=O)C3CC3c3ccccn3)cc2)cc1] | >5000* |

*cAMP assay

Example 1 of the present invention differs structurally from the compounds disclosed in WO2022/129933 and literature [C. Jin, A. M. Decker, V. H. Makhijani, J. Besheer, E. Darcq, B. L. Kieffer, R. Maitra, J Med Chem 2018, 61, 6748-6758] in that it contains a N-(2-(difluoromethoxy)ethyl)-moiety in combination with a bicyclic [-methyl-1H-indazol-7-yl]-group substituted instead of substituted monocyclic ring system and a pyrimidine ring instead of a pyridine. These structural differences unexpectedly result in Example 1 being more active on GPR88. In addition, Example 1 demonstrates a better pharmacokinetic profile than the exemplary prior art compounds.

### Use in Treatment/Method of Use

The present invention is directed to a compound which is useful in the treatment of a disease, symptom, disorder and condition wherein the agonism of GPR88 is of therapeutic benefit, including but not limited to the treatment and/or prevention of psychiatric and neurological conditions and symptoms associated with impulse control deficits and mood disorders.

Impulse control deficits are seen in addictions including substance use disorders; personality disorders such as borderline personality disorder; eating disorders such as binge eating disorder; or attention deficit hyperactivity disorder. According to a further aspect of the invention, compounds of the present invention are useful in the treatment of GPR88 related pathophysiological disturbances in arousal/wakefulness, appetite/food intake, cognition, motivated behaviours/reward, mood disorders, disorders associated with stress, anxiety, panic and symptoms associated with such disorders.

In view of their pharmacological effect, compounds of the present invention are suitable for use in the treatment or prevention of a disease, condition or associated symptom selected from the list consisting of
(1) treatment or prevention of substance abuse/dependence/seeking or addiction as well as relapse prevention (includes but not limited to drugs, such as cocaine, opiates such as morphine, barbiturates, benzodiazepines, amphetamines, nicotine/tobacco and other psychostimulants), alcoholism and alcohol-related disorders, drug abuse or addiction or relapse, tolerance to narcotics or withdrawal from narcotics;
(2) eating disorders, such as binge eating, bulimia nervosa, anorexia nervosa, other specified feeding or eating disorders, obesity, overweight, cachexia, appetite/taste disorders, vomiting, nausea, Prader-Willi-Syndrome, hyperphagia, appetite/taste disorders;
(3) attention deficit hyperactivity disorder, conduct disorders, attention problems and related disorders, sleep disorders, anxiety disorders such as generalized anxiety disorder, panic disorders, phobias, post-traumatic stress disorders, schizophrenia, Alzheimer's disease, Parkinson's disease, Huntington's disease and Gilles de la Tourette's syndrome, restless legs syndrome, dementia, dyskinesia, severe mental retardation, neurodegenerative disorders including nosological entities such as disinhibition-dementia-parkinsonism-amyotrophy complex, pallido-ponto-nigral degeneration;
(4) cognitive dysfunction in psychiatric or neurological disorder, cognitive impairments associated with schizophrenia, Alzheimer's disease and other neurological and psychiatric disorders;
(5) mood disorders, bipolar disorder, mania, depressive disorders such as treatment resistant depression and major depressive disorder, manic depression, personality disorders such as borderline personality disorder and antisocial personality disorder, aggression such as impulsive aggression, suicidality, frontotemporal dementia, obsessive compulsive disorder, delirium, affective neurosis/disorder, depressive neurosis/disorder, anxiety neurosis, dysthymic disorder (6) sexual disorders, sexual dysfunction, psychosexual disorder;
(7) impulse control disorders such as pathological gambling, trichotillomania, intermittent explosive disorder, kleptomania, pyromania, compulsive shopping, internet addiction, sexual compulsion;
(8) sleep disorders such as narcolepsy, jetlag, sleep apnea, insomnia, parasomnia, disturbed biological and circadian rhythms, sleep disturbances associated with psychiatric and neurological disorders;
(9) treatment, prevention and relapse control of impulsivity and/or impulse control deficits and/or behavioural disinhibition in any psychiatric and/or neurological condition;
(10) personality disorders such as borderline personality disorder, antisocial personality disorder, paranoid personality disorder, schizoid and schizotypal personality disorder, histrionic personality disorder, narcissistic personality disorder, avoidant personality disorder, dependent personality disorder, other specified and non-specified personality disorders;
(11) neurological diseases, such as cerebral oedema and angioedema, cerebral dementia like e.g. Parkinson's and Alzheimer's disease, senile dementia; multiple sclerosis, epilepsy, temporal lobe epilepsy, drug resistant epilepsy, seizure disorders, stroke, myasthenia gravis, brain and meningeal infections like encephalomyelitis, meningitis, as well as schizophrenia, delusional disorders, autism, affective disorders and tic disorders;
(12) Cancer, such as prostate cancer, liver cancer, lung cancer, melanoma, lung cancer non-Hodgkin's lymphoma, melanoma;
(13) Gastrointestinal diseases, involving the gastrointestinal tract, namely the esophagus, stomach, small intestine, large intestine and rectum, and the accessory organs of digestion, liver, gallbladder, and pancreas;
(14) Metabolic Syndrome or Type I and II diabetes and other metabolic disorders such as non-alcoholic steatohepatitis (NASH);
(15) Treatment of acute and chronic pain such diabetic neuropathy, polyneuropathy, low back pain, rheumatism, myalgia, carpal tunnel syndrome;
(16) Treatment of cerebrovascular diseases, such as intracerebral or subararchnoid haemor-rhage, cerbral infarction, stroke, occlusion and stenosis, cerebral atherosclerosis, cerebral amyloid angiopathy;
(17) Hypothalamic disorders such as energy balance, nutrient metabolism, hypopituitarism;
(18) Trauma and acute and chronic stress related syndrome such as stress ulceration, tachycardia, sweating, hyperventilation.

A further embodiment of the invention is therefore directed to a method for treating a psychiatric or neurological condition associated with impulse control deficits, addiction, speech delay, learning disabilities, depression, psychosis, bipolar disorder and/or emotional valence in a patient comprising administering to said patient a therapeutically effective amount of a compound of the present invention, or a pharmaceutically acceptable salt thereof.

In a more specific embodiment, the psychiatric or neurological condition to be treated is selected from the group consisting of major depressive disorder, Huntington's disease, addiction, Parkinsons's disease, attention deficit hyperactivity disorder, borderline personality disorder, post-traumatic stress disorder, attention deficit hyperactivity disorder and cognitive impairments associated with schizophrenia.

As used herein, unless otherwise noted, the terms “treating”, “treatment” shall include the management and care of human subjects or patients for the purpose of combating a disease, condition, symptom or disorder and includes the administration of the compound of the present invention to prevent the onset of the symptoms or complications, alleviate the symptoms or complications, or eliminate the disease, symptom, condition, or disorder.

As used herein, unless otherwise noted, the term “prevention” shall include (a) reduction in the frequency of one or more symptoms; (b) reduction in the severity of one or more symptoms; (c) delay or avoidance of the development of additional symptoms; and/or (d) delay or avoidance of the development of the disorder or condition.

The applicable daily dose of compounds of the present invention may vary from 0.001 to 2000 mg.

The actual therapeutically effective amount or therapeutic dose will depend on factors known by those skilled in the art such as age and weight of the patient, route of administration and severity of disease. In any case, the drug substance is to be administered at a dose, and in a manner, which allows a therapeutically effective amount to be delivered that is appropriate to the patient's condition.

### Pharmaceutical Compositions

A further embodiment of the invention is directed to a pharmaceutical composition comprising the compound of the present invention, or a pharmaceutically acceptable thereof, and a pharmaceutically acceptable adjuvant, diluent and/or carrier. Suitable pharmaceutical compositions for administering the compounds of the present invention will be apparent to those with ordinary skill in the art and include for example tablets, pills, capsules, suppositories, lozenges, troches, solutions, syrups, elixirs, sachets, injectables, inhalatives, powders. The content of the pharmaceutically active compound(s) may vary in the range from 0.1 to 95 wt.-%, preferably 5.0 to 90 wt.-% of the composition as a whole.

Suitable tablets may be obtained, for example, by mixing a compound of the present invention with known excipients, for example inert diluents, carriers, disintegrants, adjuvants, surfactants, binders and/or lubricants and pressing the resulting mixture to form tablets.

The compound described herein can be administered topically, orally, transdermally, rectally vaginally, parentally, intranasally, intrapulmonary, intraocularly, intracardially, intramuscularly, intravenously, intradermally, intraperitoneally, subcutaneously, intraspinally, sublingually, subcapsularly, intraarticularly, or by inhalation.

### Combination Therapy

Compounds according to the present invention can be combined with other treatment options known to be used in the art in connection with a treatment of any of the indications the treatment of which is in the focus of the present invention.

Among such treatment options that are considered suitable for combination with the treatment according to the present inventions are:
Antidepressants
Mood stabilizers
Antipsychotics
Anxiolytics
Antiepileptic drugs
Sleeping agents
Cognitive enhancers
Stimulants
Non-stimulant medications for ADHD
Additional psychoactive drugs.
Natural products
Tourette's Syndrom
Huntington's Disesase (HD)
Addiciton
Parkinsons's Disease (PD)
Schizophrenia
Attention Deficit Hyperactivity Disorder (ADHD)
Choreiform movements
Speech delay
Learning disabilities
Hyperkinetic movement disorders characterised by chorea and/or dystonia
Psychosis
Cognitive deficits in schizophrenia
Affective disorders
Bipolar disorder
Alzheimer's disease
Basal ganglia disorders
Digital therapies
Behavioural therapies

### Experimental Section

### HPLC-Methods:

Method Name: A
Column: Colonne acquity CSH Xselect C18 1.7 μm 2.1×30 mm
Column Supplier: Waters

| Gradient/Solvent | % Sol | % Sol | Flow | Temp |
|---|---|---|---|---|
| Time [min] | [H₂O, 0.1% TFA] | [ACN] | [mL/min] | [° C.] |
| 0.00 | 97 | 3 | 0.8 | 55 |
| 0.30 | 97 | 3 | 0.8 | 55 |
| 2.50 | 0 | 100 | 0.8 | 55 |
| 2.80 | 0 | 100 | 0.8 | 55 |
| 3.00 | 97 | 3 | 0.8 | 55 |
| 3.50 | 97 | 3 | 0.8 | 55 |

### Preparation of Compounds of the Present Invention

### Synthesis of Acids Intermediates

### (1R,2R)—N-(2-(difluoromethoxy)ethyl)-N-(4-(4-isopropoxyphenyl)-1-methyl-1H-indazol-7-yl)-2-(pyrimidin-4-yl) cyclopropane-1-carboxamide

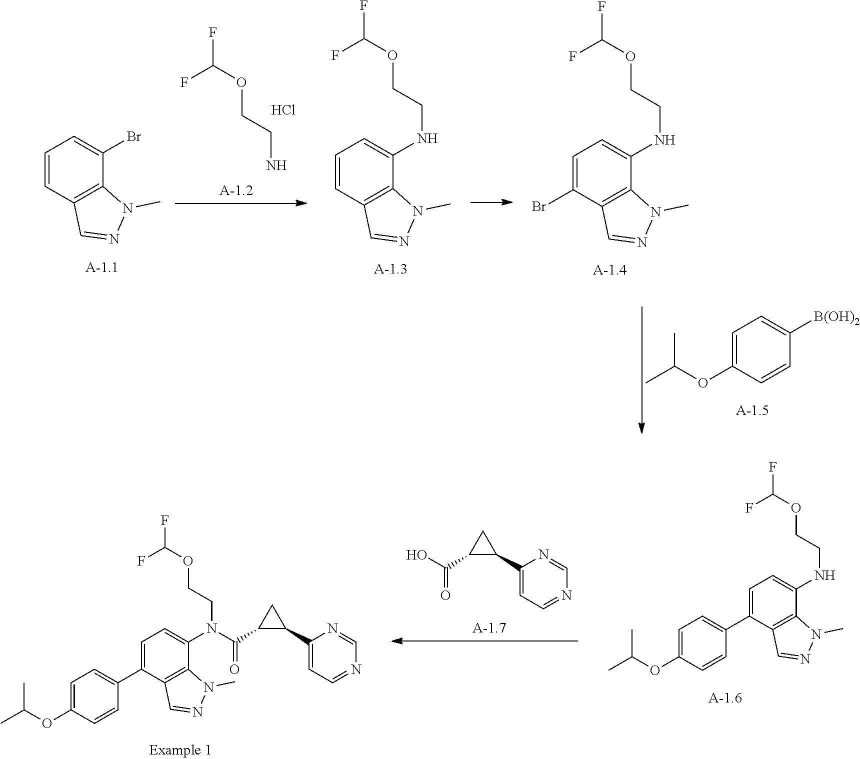

Step 1: A suspension of A-1.1 (200 mg, 0.95 mmol, 1.0 Eq), A-1.2 hydrochloride (210 mg, 1.42 mmol, 1.5 Eq) and cesium carbonate (1.08 g, 3.32 mmol, 3.5 Eq) in 1,4-dioxane (4 mL) was sparged with argon for 10 min. BrettPhosPdG4 (65 mg, 0.071 mmol, 0.075 Eq) was then added in one portion. The reaction was heated at 90° C. for 18 h. After cooling, the reaction mixture was filtered over celite, the cake was washed with EA (50 mL) and the filtrate was concentrated to dryness. The crude residue was purified by flash chromatography (20 μm, CyHex 100% to CyHex/EA 30:70) to obtain an orange solid (198 mg, 87%). ¹H NMR (400 MHz, DMSO-d₆) δ_{H} ppm 7.84 (s, 1H), 7.04 (dd, 1H, J=8.0, J=0.6 Hz), 6.91 (t, 1H, J=8.0 Hz), 6.74 (t, 1H, J_{H-F}=75.9 Hz), 6.51-6.47 (m, 1H), 5.45 (t, 1H, J=5.7 Hz), 4.27 (s, 3H), 4.09 (t, 2H, J=5.7 Hz), 3.39 (q, 2H, J=5.7 Hz); ESI-MS: 242.2 [M+H]⁺; HPLC (Rt): 4.76 min (method A).

Step 2: NBS (728 mg, 4.09 mmol, 1.05 eq.) was added portion wise to a 0° C. solution under Ar of A-1.3 (940 mg, 3.90 mmol, 1.0 eq.) in DMF (45 mL). The reaction was stirred at RT for 1 hr, diluted in water (150 mL), and was then extracted with EA (3×150 mL). The organic layer was washed with brine, dried over sodium sulfate, filtered, then concentrated to dryness. The crude was purified by flash column chromatography (CyHex 100% to CyHex/EA 40:60) to afford the title compound as a red solid (1.0 g, 80%). ¹H NMR (400 MHz, DMSO-d₆) δ_{H} ppm 7.80 (s, 1H), 7.04 (d, 1H, J=8.1 Hz), 6.73 (t, 1H, J_{H-F}=75.9 Hz), 6.44 (d, 1H, J=8.1 Hz), 5.61 (t, 1H, J=5.7 Hz), 4.27 (s, 3H), 3.38 (q, 2H, J=5.7 Hz); ESI-MS: 322.1 [M81 [Br]+H]⁺; HPLC (Rt): 5.21 min (method A).

Step 3: A solution of A-1.4 (70 mg, 0.22 mmol, 1.0 eq.) and A-1.5 (59 mg, 0.33 mmol, 1.5 eq.) in 1.2 M K₂CO₃ (aq.1.2 M, 364 μL, 0.44 mmol, 2.0 eq) in 1,4-dioxane (3 mL) was sparged with Ar for 15 min. PdCl₂dppf (16 mg, 0.022 mmol, 0.1 eq.) was added in one portion. The reaction was finished in 1 hr at 100° C. The crude was purified by flash column chromatography (20 μM, DCM 100% to DCM/EA 30:70) to obtain the title compound as a white solid (57 mg, 69%). 1H NMR (400 MHz, DMSO-d₆) δ_{H} ppm 7.79 (s, 1H), 7.53-7.48 (m, 2H), 7.22-6.96 (m, 3H), 6.76 (t, 1H, J_{H-F}=76.2 Hz), 6.58 (d, 1H, J=7.8 Hz), 5.52 (t, 1H, J=5.7 Hz), 4.64 (hept, 1H, J=6.3 Hz), 4.32 (s, 3H), 4.11 (t, 2H, J=6.3 Hz), 3.44 (q, 2H, J=6.3 Hz), 1.29 (d, 6H, J=6.3 Hz); ESI-MS: 376.3 [M+H]⁺; HPLC (Rt): 3.84 min (method A).

Step 4: POCl₃ (27 μL, 0.29 mmol, 2.0 eq.) was added dropwise to a 0° C. solution of A-1.6 (55 mg, 0.15 mmol, 1.0 eq.) and A-1.7 (39 mg, 0.22 mmol, 1.5 eq.) in pyridine (2 mL) under Ar atmosphere. The reaction was stirred at 25° C. for 18 hr. The mixture was hydrolyzed at 0° C. with NaOH 1N, and extracted with EA. The combined organic layers were dried over sodium sulfate, filtered, then concentrated to dryness. The crude was purified by flash column chromatography (20 μM, DCM 100% to DCM/EA 0:100) to obtain two isolable atropoisomers of the title compound as white foams (major, 54 mg, 71%, minor, 14 mg, 18%). Data Example 1 f (major atropoisomer): ¹H NMR (400 MHz, DMSO-d₆) δ_{H} ppm d 8.73 (d, 1H, J=1.2 Hz), 8.57 (d, 1H, J=5.1 Hz), 8.21 (s, 1H), 7.62-7.52 (m, 3H), 7.25 (d, 1H, J=7.5 Hz), 7.09-7.01 (m, 3H), 6.68 (t, 1H, J_{H-F}=76.2 Hz), 4.74 (hept, 1H, J=6.0 Hz), 4.42 (td, 1H, J=14.2 Hz, 6.0 Hz), 4.07 (s, 3H), 4.05-3.97 (m, 2H), 3.58 (td, 1H, J=14.2 Hz, 6.0 Hz), 2.66-2.60 (m, 1H), 1.89-1.83 (m, 1H), 1.62-1.56 (m, 1H), 1.35-1.27 (m, 7H); ¹³C NMR (150 MHz, DMSO-d₆) δ_{C} ppm 171.0, 167.3, 158.0, 157.5, 156.3, 136.0, 134.8, 132.5, 130.1, 129.5 (2C), 128.1, 124.3, 123.3, 120.2, 119.5, 116.7, 115.9 (2C), 69.3, 61.3, 48.4, 37.4, 25.7, 24.9, 21.8 (2C), 17.5; ESI-HRMS: calcd for C₂₈H₃₀F₂O₃N₅: 522.2311, found: 522.2310 [M+H]⁺; ESI-MS: 522.3 [M+H]⁺; HPLC (Rt): 5.35 min (method A); Data minor atropisomer: 1H NMR (400 MHz, DMSO-d₆) δ_{H} ppm d 8.60 (d, 1H, J=1.1 Hz), 8.48 (d, 1H, J=5.1 Hz), 7.96 (s, 1H), 7.57 (d, 2H, J=8.5 Hz), 7.44 (d, 1H, J=8.1 Hz), 7.38 (dd, 1H, J=5.1, 1.1 Hz), 7.22 (d, 1H, J=7.5 Hz), 7.06 (d, 2H, J=8.5 Hz), 6.70 (t, 1H, J_{H-F}=76.2 Hz), 4.70 (hept, 1H, J=6.5 Hz), 4.39 (td, 1H, J=14.2 Hz, 6.0 Hz), 4.13-4.00 (m, 2H), 3.92 (s, 3H), 3.71 (td, 1H, J=14.2 Hz, 6.0 Hz), 2.67-2.58 (m, 1H), 1.90-1.81 (m, 1H), 1.61-1.54 (m, 1H), 1.31-1.24 (m, 7H); ¹³C NMR (150 MHz, DMSO-d₆) δ_{C} ppm 171.0, 166.9, 157.7, 157.5, 156.2, 136.0, 134.7, 132.2, 130.1, 129.5 (2C), 127.7, 124.0, 123.5, 119.7, 119.6, 116.8, 115.9 (2C), 69.3, 61.5, 48.4, 37.2, 25.8, 25.1, 21.8 (2C), 16.8; ESI-MS: 522.4 [M+H]⁺; HPLC (Rt): 5.310 min (method A)

1. The compound having the following structure:

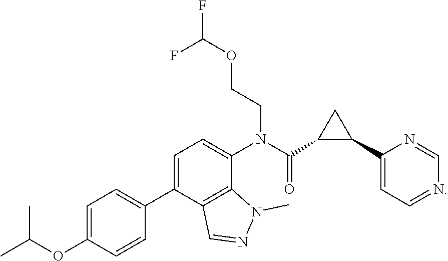 [SMILES: CC(C)Oc1ccc(-c2ccc(N(CCOC(F)F)C(=O)[C@@H]3C[C@H]3c3ccncn3)c3c2cnn3C)cc1]

2. A pharmaceutically acceptable salt of the compound according to claim 1.

3. A pharmaceutical composition comprising the compound according to claim 1, or a pharmaceutically acceptable salt thereof, and a pharmaceutically acceptable adjuvant, diluent and/or carrier.

4. A method for treating a psychiatric or neurological condition associated with impulse control deficits, addiction, speech delay, learning disabilities, depression, psychosis, bipolar disorder and/or emotional valence in a patient comprising administering to said patient a therapeutically effective amount of a compound according to claim 1, or a pharmaceutically acceptable salt thereof.

5. The method according to claim 4, wherein the psychiatric or neurological condition to be treated is selected from the group consisting of major depressive disorder, Huntington's disease, addiction, Parkinsons's disease, attention deficit hyperactivity disorder, borderline personality disorder, post-traumatic stress disorder, attention deficit hyperactivity disorder and cognitive impairments associated with schizophrenia.
